(19) United States
(12) Patent Application Publication
(10) Pub. No.: US 2013/0030361 A1
(43) Pub. Date: Jan. 31, 2013

(54) COATED MEDICAL IMPLANT

(71) Applicants: Maria Astrand, Storvreta (SE); Hakan Engqvist, Osthammar (SE)

(72) Inventors: Maria Astrand, Storvreta (SE); Hakan Engqvist, Osthammar (SE)

(73) Assignee: Sandvik Intellectual Property AB, Sandviken (SE)

(21) Appl. No.: 13/640,507

(22) PCT Filed: Apr. 12, 2011
(86) PCT No.: PCT/SE11/50444

§ 371 Date: Oct. 11, 2012

(30) Foreign Application Priority Data
Apr. 12, 2010 (SE) 1000362-2
Apr. 12, 2010 (SE) 1000363-0

(51) Int. Cl. A61M 5/00; C23C 16/40

(52) U.S. Cl. 604/93.01; 427/2.26

(57) ABSTRACT

The present invention relates to a coated medical implant wherein the coating comprises an amorphous Me oxide layer or an (Al, Me) oxide layer deposited by physical vapor deposition, and wherein Me can be one or more of the elements of Ti, Si, Cr, Hf, Zr, Ta or Nb. The PVD layer does not have any bioactive/osseointegrating properties at temperatures below 45° C. This makes it easy to remove the implant after it has being inserted in an animal or human body, e.g. when used for fixating fractures. The coating may further comprise a calcium phosphate layer, preferably a hydroxyapatite layer, which is grown on the PVD layer using a biomimetic process. The coating may be loaded with a releasable agent such as a pharmaceutical agent, an ion or a bio molecule. The present invention also relates to a method for forming the coated medical implant.

### TECHNICAL FIELD

The present invention relates to a coated medical implant comprising a coating layer deposited by physical vapor deposition and optionally also a calcium phosphate coating layer such as a hydroxyapatite grown on the PVD layer. The coating may be loaded with a releasable agent such as a pharmaceutical agent, an ion or a bio molecule.

### BACKGROUND

Applying coatings to medical bone implants such as hip joints etc. is well known in the art. Coatings are applied for different reasons, e.g. increased wear resistance, improved biocompatibility and/or bioactivity.

By biocompatible is meant that the implant does not have any toxic or injurious effects on biological tissue. For some implant surfaces, e.g. those that are meant to bond with bone tissue, it is of importance to have bioactivity. By bioactive is meant that the material is capable of biochemically bonding to the bone tissue. A common method to verify bioactivity is to soak the implant surface in simulated body fluid (SBF). If hydroxyapatite is formed on the soaked implant surface, the implant surface is regarded as bioactive.

Vapor deposition processes such as chemical vapor deposition (CVD) and physical vapor deposition (PVD) are common techniques for coating semiconductors, optical surfaces, cutting tools etc. These techniques have also been used to coat implant surfaces where a corrosion barrier or an increased wear resistance is wanted, e.g. at the articular interface of a hip joint.

Implants coated with TiO₂ coatings deposited using PVD are known in the art.

WO 2009/091331 describes a method for depositing a crystalline TiO₂ coating onto a bone anchored implant by PVD techniques. The crystalline TiO₂ coating has bioactive properties and will therefore anchor to bone tissue.

Sometimes it is also beneficial to load an implant surface with active substances such as antibiotics etc. before insertion into the body.

WO 2008/056323 discloses a surgical implant composite material with a thin film coating made of crystalline TiO₂. The thin film coating may be loaded with a releasable agent comprising an active pharmaceutical agent, an ion or a bio molecule. The crystalline TiO₂ is bioactive and therefore a biomimetic coating comprising e.g. hydroxyapatite or other calcium phosphate can be grown thereon. As an alternative to loading of the thin film coating the biomimetic coating can be loaded with the releasable agent.

However, sometimes when implants such as screws, nails and plates are inserted into the body, e.g. to fixate bone fractures, the implants have to be removed after a number of weeks when the fracture has healed. A too strong anchoring will make the removal more difficult without damaging the bone.

### SUMMARY OF THE INVENTION

It is an object of the present invention to provide a medical implant that easily can be removed after being inserted into the body. This object is achieved with a medical implant and a method for producing such as defined in the independent claims.

A coated medical implant in accordance with one embodiment of the invention comprises a PVD layer composed of an (Al, Me) oxide. When soaked in a phosphate buffered saline solution, which commonly is referred to as a simulated body fluid, of about pH 7 and temperature below 45 ° C. for a period of 1 week substantially no hydroxyapatite is formed on the PVD layer, i.e. the PVD layer is bio inert.

A coated medical implant in accordance with another embodiment of the invention comprises a PVD layer composed of an (Al, Me) oxide or an amorphous (Ti, Me) oxide. When soaked in a phosphate buffered saline solution, which commonly is referred to as a simulated body fluid, of about pH 7 and temperature below 45° C. for a period of 1 week substantially no hydroxyapatite is formed on the PVD layer, i.e. the PVD layer is bio inert.

A method for producing a medical implant in accordance with one embodiment of the invention comprises the steps of providing a implant body and depositing a coating on the implant body. The step of depositing comprises depositing a PVD layer on the implant body, wherein the PVD layer is composed of an (Al, Me) oxide or an amorphous (Ti, Me) oxide. The PVD layer is bio inert in that substantially no hydroxyapatite is formed on the PVD layer when soaked in a phosphate buffered saline solution of about pH land temperature below 45° C. for a period of 1 week.

The element Me of said (Ti, Me) oxide is preferably one or more of the elements Si, Cr, Hf, Zr, Ta and Nb, and it serves to disturb the formation of a crystalline (Ti, Me) oxide layer during deposition. In one embodiment Me is Si, preferable in an amount of about 10 at-%.

In said (Al, Me) oxide Me is one or more of the elements Ti, Si, Cr, Hf, Zr, Ta and Nb. One effect of adding these elements is that the crystallinity of the (Al, Me) oxide layer can be controlled. Further, these elements may contribute to the formation of composite coatings comprising two or more oxides of different composition and/or phase.

The deposition of the PVD layer may be controlled to obtain a porous PVD layer.

In one embodiment of the invention the coating further comprises a calcium phosphate layer, preferably a hydroxyapatite layer, grown on the PVD layer. Growth is preferably performed by a biomimetic process where the PVD layer is soaked in a simulated body fluid, such as a phosphate buffered saline solution. The growth comprises two phases including nucleation of the calcium phosphate layer on the surface of the PVD layer and continued growth from the nucleated surface layer. Since the PVD layer is bio inert at temperatures below 45° C., growth of the calcium phosphate layer, at least in the nucleation phase, is performed at an elevated temperature above 45° C., preferably 45° C. to 90° C., more preferably 55° C. to 65° C., most preferably about 60° C. The growth of the calcium phosphate layer may be controlled to obtain a porous calcium phosphate layer.

When having a porous layer in the coating of the medical implant it may be loaded with a releasable agent, such as an active pharmaceutical agent, an ion or a bio molecule. In one embodiment of the invention a releasable agent is loaded in a porous calcium phosphate layer on the PVD layer. In another embodiment of the invention the PVD layer is porous and the releasable agent is loaded in the PVD layer, and optionally, if a porous calcium phosphate layer is grown on the PVD, also in the porous calcium phosphate layer.

Thanks to the inertness of the PVD layer of embodiments of the invention the medical implant being coated with this PVD layer can easily be removed even after several weeks in human bone.

Moreover, by way of example, a medical implant comprising a calcium phosphate layer grown on the inert PVD layer, as described above, can be used in temporary fixation of bone fractures. The calcium phosphate layer may during the fixation be at least partly resorbed and thereby contribute to form new bone tissue adjacent the medical implant. However, in contrast to prior art implants like those described in the background the medical implant will be easier to remove since the calcium phosphate layer and/or the bone tissue formed does not adhere too strongly to the PVD layer.

Yet another advantage is that a coating loaded with a releasable agent in accordance with embodiments of the invention may be advantageous for targeted or controlled release in vivo.

Other objects, advantages and novel features of the invention will become apparent from the following detailed description of the invention when considered in conjunction with the accompanying drawings and claims.

### BRIEF DESCRIPTION OF DRAWINGS

Embodiments of the invention will now be described with reference to the accompanying drawings, wherein

FIG. 1 shows HA grown at 60° C. on Ti-plates coated with a (Ti_{0.9}Si_{0.1})O₂ layer, and

FIG. 2 shows HA grown at 60° C. on Ti-plates coated with a Al₂O₃ layer.

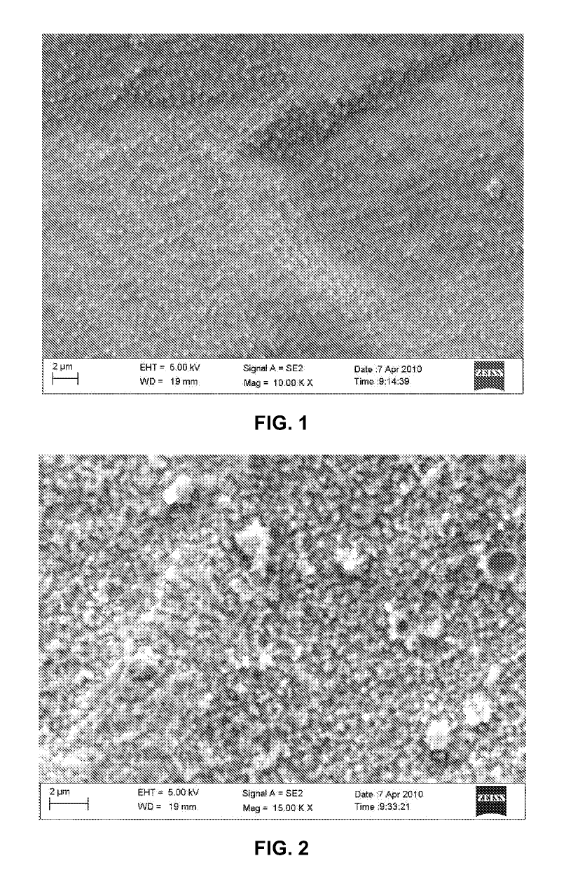

### DETAILED DESCRIPTION OF THE INVENTION

A coated medical implant in accordance with one embodiment of the present invention comprises a PVD layer composed of an (Al, Me) oxide or an amorphous (Ti, Me) oxide. When soaked in a phosphate buffered saline solution (PBS), which commonly is referred to as a simulated body fluid (SBF), of about pH 7 and temperature of below 45° C. for a period of 1 week substantially no hydroxyapatite is formed on the PVD layer, i.e. the PVD layer is bio inert. By substantially no HA formation is meant that the PVD layer is covered by less than 10% of HA, preferably less than 5% after being soaked in the PBS under the above-mentioned conditions.

In this test of the bioactivity of the PVD layer the phosphate buffered saline solution comprises CaCl₂, MgCl₂, KCl, KH₂PO₄, NaCl and Na₂HPO₄ in an ion composition and concentration similar to those of blood plasma, preferably the phosphate buffered saline solution is D8662- Dulbecco's Phosphate Saline from Sigma-Aldrich.

By amorphous is herein meant that no X-ray diffraction peaks can be found in X-ray crystallography using the following parameters: voltage 45 kV, current 40 mA, scan range 20-70°, no monocromator, sollerslit 0.02°, fixed divergence slit ¼°, fixed anti scatter slit ¼°, Ni-filter, step length 0.008°, scan speed 0.03°/s, step time 36 s and continued scan.

By medical implant is herein meant any implant that is to be inserted into the animal or human body. Examples are orthopedic and dental prostheses and fracture fixation implants such as nails, screws, plates etc.

In one embodiment of the present invention the coating comprises a PVD layer of an amorphous (Ti, Me) oxide where Me is one or more of the elements Si, Cr, Hf, Zr, Ta or Nb. The amount of these elements is substantial and cannot be regarded as impurities. The addition of the elements serves to disturb the formation of a crystalline (Ti, Me) oxide layer during deposition.

In one embodiment the amorphous (Ti,Me) oxide is an (Ti,Si) oxide. The Si has to be provided in an amount such that the Si disturbs the formation of crystalline Ti oxide during deposition. This can easily be verified by X-ray diffraction. In one embodiment the (Ti,Si) oxide comprises about 10 at-% Si.

In another embodiment of the present invention the coating comprises a PVD layer of a crystalline or an amorphous (Al, Me) oxide, e.g. Al₂O₃, where Me is one or more of the elements Ti, Si, Cr, Hf, Zr, Ta or Nb. By the addition of said one or more elements a composite PVD layer may be formed.

The thickness of the PVD layer according to the present invention is >3 nm, preferably >5 nm and most preferably >10 nm, but <8000 nm, preferably <3000 nm, and most preferably <1000 nm.

By controlling the deposition of the PVD layer the PVD layer can be made porous, which makes it suitable for loading with a releasable agent.

In one embodiment of the present invention, the porous PVD layer of the medical implant is loaded with a releasable agent, having desired functional properties, e.g. an active pharmaceutical drug, bio molecule, ions, or combinations thereof. Specific examples of releasable agents include, but are not limited to, antibiotics, e.g., gentamicin, such as gentamicin sulfate, and other aminoglycosides such as amikacin, kanamycin, neomycin, netilmicin, paromomycin, streptomycin, tobramycin, and apramycin, proteins such as bone morphogenesis proteins, peptides, bisphosphonates, opioids, opiates, vitamins, anti-cancer drugs, iodine, Ag, combinations thereof, and the like.

In one embodiment of the present invention the coating further comprises an additional layer formed on the PVD layer. The thickness of the this additional layer is >3 nm, preferably >5 nm and most preferably >10 nm, but <10 μm, preferably <5 μm, and most preferably <2 μm.

In one embodiment of the invention the additional layer is a calcium phosphate layer, e.g. a hydroxyapatite layer, grown by a biomimetic process on the PVD layer. The growth of the calcium phosphate layer can be controlled to obtain a porous coating.

Hydoxyapatite (Ca₁₀(PO₄)₆(OH)₂ is mineral that is widely used in medical applications due to its similarity with the mineral components of bone and teeth. Hydroxyapatite is a calcium phosphate. As appreciated by a person skilled in the art a calcium phosphate layer in accordance with the invention may include other calcium phosphates than hydroxyapatite.

In one embodiment the additional layer formed on the PVD layer is an outermost porous coating. The outermost porous coating may be loaded with a releasable agent. Examples of such releasable agents have been mentioned above for the loading of the PVD layer.

In one embodiment the outermost porous coating is a resorbable polymer comprising polyactic acids, propylene fumarate, chitosan or cyclodextrin, or combinations thereof.

The implant bulk material can be any material suitable for implants. Examples of such materials are titanium, titanium-alloys, cobalt, cobalt alloys, tool steel, stainless steel and Co—Cr—Mo-alloys. The bulk material may have been pre-treated before depositing the PVD layer, e.g. by pre-coating with other coating materials or by thermal treatments to e.g. oxidize the bulk material or the pre-coated coating. The bulk material and any surface layer formed by pre-treatment forms a body of the implant.

A method of forming a coated medical implant in accordance with one embodiment of the present invention comprises the steps of:
providing an implant body; and
depositing a coating on the implant body comprising depositing a PVD layer on the implant body, wherein the PVD layer is composed of an (Al, Me) oxide or an amorphous (Ti, Me) oxide being bio inert in that substantially no hydroxyapatite is formed on the PVD layer when soaked in a phosphate buffered saline solution of about pH 7 and temperature below 45° C. for a period of 1 week.

In one embodiment of the invention the method further comprises depositing a calcium phosphate layer, such as a HA layer, on the PVD layer by soaking the PVD layer in a phosphate buffered saline solution of pH 6-8, preferably about pH 7, and a temperature of at least 45° C., preferably 45° C. to 90° C., more preferably 55° C. to 65° C., most preferably about 60° C. for a sufficient time to form the desired thickness. The temperature may be the same during the whole growth but may also be changed after an initial nucleation period.

Biomimetic processes for depositing calcium phosphate coatings are known in the art. Different phosphate buffered saline solutions are known. However, in prior art processes the implant body comprises a bioactive material on the surface thereof in order to enable growth of the calcium phosphate on the surface and to get an acceptable adhesion between the bioactive material and the calcium phosphate.

The thickness of the calcium phosphate layer prepared according to the above method can be controlled in thickness between 10 nm and 100 μm. This can be controlled by soaking time, temperature and composition of the phosphate buffered saline solution. Deposition times range from 1 day to several weeks.

PVD techniques suitable for forming the PVD layer of the present invention are any PVD technique known in the art such as any one of cathodic arc evaporation, magnetron sputtering, or e-beam evaporation, preferably cathodic arc evaporation. The process parameters are those commonly used in the art of PVD deposition. The deposition time varies depending on the chosen PVD technique and the desired PVD layer thickness. As explained above deposition of the PVD layer and/or growth of the calcium phosphate layer may be controlled to obtain porosity in the coating.

The method may further comprise loading of this porous coating with the same releasable agents as has been mentioned for the loading of a porous PVD layer above. The coating is loaded with a releasable agent using any method known in the art, e.g. soaking, vacuum impregnating, absorption loading, solution loading, evaporating loading, solvent loading, air suspension coating techniques, precipitation techniques, spray coagulation techniques, or combinations thereof.

An outermost porous coating, such as the resorbable polymer discussed above, can be deposited by any suitable technique such as solution deposition, melting or spraying onto the implant followed by drying or solidifying.

### Example 1

Titanium plates, 20×20×1 mm, were coated with (Ti_{0.9}Si_{1.0})O₂ using cathodic arc evaporation.

The Ti-plates were ultrasonically cleaned in 2-propanol for 10 minutes, rinsed in deionized water for 10 minutes dried in hot air before being mounted on a 2-fold rotating table inside a PVD chamber with (Ti, Si) sources with 10% Si and 90% Ti. Prior to deposition the Ti-plates were heated to the deposition temperature of 320° C., followed by Ar-ion etching to remove any surface contaminants. During deposition oxygen was introduced into the chamber at a flow of 800 sccm and substrate bias was −60 V. The deposition time was 20 minutes, resulting in a 0.5 μm thick (Ti_{0.9}Si_{0.1})O₂ layer on the Ti-plates.

### Example 2-5

Titanium plates, 20×20×1 mm, were coated in the same way as in Example 1, but with different deposition conditions with respect to one or more of deposition temperature, bias and oxygen flow as can be seen in Table 1.

### Example 6

To evaluate the bioactivity, i.e. the ability to form hydroxyapatite (HA) on the (Ti_{0.9}Si_{0.1})O₂coatings, from the coated Ti-plates of Examples 1-5, as well as two reference sample plates, Ref 1 being Ti-plates coated with crystalline TiO₂ (anatase) and Ref. 2 being uncoated Ti-plates covered with native oxide, were soaked in Dulbecoo's phosphate buffered saline D8662 from Sigma-Aldrich (D-PBS).

Conical plastic tubes were filled with 40 ml D-PBS and preheated to 37° C. and 60° C., respectively. The coated Ti-plates of examples 1-5 and reference sample plates 1-2 were ultrasonically cleaned in isopropanol and deionized water, dried in nitrogen gas and vertically placed in the preheated tubes. The tubes were kept at fixed temperature, .e. 37° C. and 60° C., respectively, for five days in an incubator. After five days the plates were rinsed with deionized water and dried with nitrogen.

The presence of HA on the plates was visually determined by a scanning electron microscope (SEM) and Glow Discharge Optical Emission Spectroscopy (GDOES) and graded as growth or no growth. By “growth” is herein meant that the HA layer is smooth, i.e. a conformal coating, and covers the whole plate surface. By “no growth” is meant that no HA growth could be observed. The thickness of the HA layer in Examples 1-5 was about 0.15 μm. The results are summarized in Table 1.

TABLE 1

| | O₂ flow | Deposition | Substrate | | | |
|---|---|---|---|---|---|---|
| Sample | (sccm) | temp. (° C.) | bias (V) | Coating | 37° C. | 60° C. |
| Ex. 1 | 800 | 320 | −60 | (Ti_{0.9}Si_{0.1})O₂ | no growth | growth |
| Ex. 2 | 800 | 560 | −60 | (Ti_{0.9}Si_{0.1})O₂ | no growth | n.a. |
| Ex. 3 | 800 | 320 | −20 | (Ti_{0.9}Si_{0.1})O₂ | no growth | n.a. |
| Ex. 4 | 800 | 320 | −120 | (Ti_{0.9}Si_{0.1})O₂ | no growth | n.a. |
| Ex. 5 | 400 | 320 | −60 | (Ti_{0.9}Si_{0.1})O₂ | no growth | n.a. |
| Ref. 1 | 800 | 320 | −60 | anatase TiO₂ | growth | growth |
| Ref. 2 | n.a. | n.a. | n.a. | native oxide | no growth | growth |

### Example 7

Titanium plates, 20×20×1 mm, were coated with Al₂O₃ using pulsed magnetron sputtering.

The Ti-plates were ultrasonically cleaned in 2-propanol for 10 minutes, rinsed in deionized water for 10 minutes and dried in hot air before being mounted on a 3-fold rotating table inside a PVD chamber with pure Al sources. Prior to deposition, the Ti-plates were heated to the deposition temperature of 570° C., followed by Ar-ion etching to remove any surface contaminants. Deposition was performed for 2.5 hours resulting in a 0.5 μm thick Al₂O₃ layer. During deposition argon and oxygen gas was introduced in the chamber.

### Example 8

Titanium plates, 20×20×1 mm, were coated with Al₂O₃ using pulsed magnetron sputtering.

The Ti-plates were ultrasonically cleaned in 2-propanol for 10 minutes, rinsed in deionized water for 10 minutes and dried in hot air before being mounted on a 2-fold rotating table inside a PVD chamber comprising pure Al sources. Prior to deposition, the Ti-plates were heated to the deposition temperature of 570° C., followed by Ar-ion etching to remove any surface contaminants. Deposition was performed for 2.5 hours resulting in a 1 μm thick Al₂O₃ layer on the Ti-plates. During deposition argon and oxygen gas was introduced in the chamber.

### Example 9

To evaluate the bioactivity, i.e. the ability to form hydroxyapatite (HA) on Al₂O₃, the coated Ti-plates of examples 7 and 8, as well as two reference sample plates, Ref 3 being Ti-plates coated with crystalline TiO₂ (anatase) deposited using cathodic arc evaporation and Ref 4 being uncoated Ti-plates covered with native oxide, were soaked in Dulbecoo's phosphate buffered saline D8662 from Sigma-Aldrich (D-PBS).

Conical plastic tubes were filled with 40 ml D-PBS and preheated to 37° C. and 60° C., respectively. The coated Ti-plates of examples 7-8 and reference sample plates 3-4 were ultrasonically cleaned in isopropanol and deionized water, dried in nitrogen gas and vertically placed in the preheated tubes. The tubes were kept at fixed temperature, i.e. 37° C. and 60° C., respectively, for five days in an incubator. After five days the plates were rinsed with deionized water and dried with nitrogen.

The presence of HA on the plates was visually determined by a scanning electron microscope (SEM) and Glow Discharge Optical Emission Spectroscopy (GDOES) and graded as growth or no growth. By “growth” is herein meant that the HA layer is smooth, i.e. a conformal coating, and covers the plate surface. By “no growth” is meant that no HA growth could be observed. The thickness of the HA layer in Examples 7-8 was about 0.1 μm. The results are summarized in Table 2.

TABLE 2

| | Coating | | | |
|---|---|---|---|---|
| Sample | method | Coating | 37° C. | 60° C. |
| Example 7 | PVD | Al₂O₃ | no growth | growth |
| Example 8 | PVD | Al₂O₃ | no growth | growth |
| Ref. 3 | PVD | anataseTiO₂ | growth | growth |
| Ref. 4 | n.a. | native oxide | no growth | growth |

While the invention has been described in connection with various exemplary embodiments, it is to be understood that the invention is not to be limited to the disclosed embodiments, on the contrary, it is intended to cover various modifications and equivalent arrangements within the appended claims.

1. A medical implant, comprising:
a coating, the coating including a PVD layer, wherein the PVD layer is selected from the group of (Al, Me) oxide and amorphous (Ti, Me) oxide and wherein, substantially no hydroxyapatite is formed on the PVD layer when soaked in a phosphate buffered saline solution of about pH 7 and a temperature below 45° C. for a period of 1 week.

2. The medical implant according to claim 1, wherein the PVD layer is an outermost layer of the coating.

3. The medical implant according to claim 1, wherein Me is one or more of the elements Si, Cr, Hf, Zr, Ta and Nb.

4. The medical implant according to claim 1, wherein the PVD layer is composed of an amorphous (Ti,Si) oxide.

5. The medical implant according to claim 1, wherein the (Al, Me) oxide is amorphous.

6. The medical implant according to claim 1, wherein the (Al, Me) oxide is crystalline.

7. The medical implant according to claim 1, wherein the (Al, Me) oxide comprises one or more of the elements Ti, Si, Cr, Hf, Zr, Ta or Nb.

8. The medical implant according to claim 1, wherein the (Al, Me) oxide is Al₂O₃.

9. The medical implant according to claim 1, wherein said (Al, Me) oxide is a composite composed of at least two components having different composition and/or phase.

10. The medical implant according to claim 1, wherein the PVD layer is porous.

11. The medical implant according to claim 10, wherein the PVD layer is loaded with a releasable agent.

12. The medical implant according to claim 11, wherein the releasable agent is selected from the group of an active pharmaceutical agent, an ion or a bio molecule.

13. The medical implant according to claim 1, wherein the coating further comprises a calcium phosphate layer grown on the PVD layer.

14. The medical implant according to claim 13, wherein the calcium phosphate layer is composed of hydroxyapatite.

15. The medical implant according to claim 13, wherein the calcium phosphate layer is porous.

16. The medical implant according to claim 13, wherein the calcium phosphate layer is loaded with a releasable agent.

17. The medical implant according to claim 16, wherein the releasable agent is selected from the group of an active pharmaceutical agent, an ion or a bio molecule.

18. A method for producing a medical implant comprising the steps of:
providing an implant body; and
depositing a coating on the implant body comprising depositing a PVD layer on the implant body, wherein the PVD layer is composed selected from the group of (Al, Me) oxide or amorphous (Ti, Me) oxide such that substantially no hydroxyapatite is formed on the PVD layer when soaked in a phosphate buffered saline solution of about pH 7 and a temperature below 45° C. for a period of 1 week.

19. The method according to claim 18, wherein the step of depositing further comprises depositing a calcium phosphate layer on the PVD layer by soaking the PVD layer in a phosphate buffered saline solution of pH 6-8 and of at least 45 ° C.

20. The method according to claim 18, further comprising loading the coating with a releasable agent.
